(19) United States
(12) Patent Application Publication
(10) Pub. No.: US 2024/0307660 A1
(43) Pub. Date: Sep. 19, 2024

(54) MULTICHAMBERED DEVICE PORT AND BRAIDED SHEATH FOR A BALLOON CARDIOVASCULAR INSERTION DEVICE

(71) Applicant: East End Medical LLC, Miramar, FL (US)

(72) Inventors: Brijeshwar S. Maini, West Palm Beach, FL (US); Eddy Segura, Miramar, FL (US); Gun Hee Lee, Pinecrest, FL (US); Abdul-Jalil Qadir, Pembroke, FL (US)

(21) Appl. No.: 18/606,556

(22) Filed: Mar. 15, 2024

Related U.S. Application Data
(60) Provisional application No. 63/452,813, filed on Mar. 17, 2023.

(51) Int. Cl. A61M 25/01; A61M 25/00; A61M 39/06

(52) U.S. Cl. CPC A61M 25/0136; A61M 25/003; A61M 25/0045; A61M 25/005; A61M 39/06; A61M 2025/0031; A61M 2039/062; A61M 2210/12

(57) ABSTRACT

A cardiovascular insertion device to achieve safe, accurate and effective access to tissue, such as coronary, cardiac and peripheral vascular structures. The cardiovascular insertion device includes a multichambered device port on its proximal end and a positioning balloon on its distal end with a sheath defining a sheath lumen therebetween. The multichambered device port including a plurality of hermetically sealed chambers. The sheath includes an inner layer, a braid layer, and an outer layer. A balloon lumen and pull wire lumen extend within the braid layer.

### FIELD

The present disclosure is directed to a balloon cardiovascular insertion device and, more specifically, to a multichambered device port connected to the cardiovascular insertion device handle and a braided sheath for securing a lumen along the cardiovascular insertion device sheath.

### BACKGROUND

Cardiovascular catheterization is a medical procedure in which a long thin tube or catheter is inserted through an artery or a vein into specific areas of the cardiovascular system for diagnostic or therapeutic purposes. More specifically, cardiovascular chambers, vessels and valves may be catheterized. Cardiovascular catheterization may be used in procedures such as coronary angiography, left ventricular angiography and peripheral vascular angiography. Cardiovascular angiography facilitates visualization of the vessels and finding of potential blockages by taking X-ray images of a patient who has received a dye (contrast material) injection into a catheter previously injected in an artery or a vein. Left ventricular angiography enables examination of the left-sided heart chambers and the function of the left sided valves of the heart and may be combined with cardiovascular angiography. Cardiovascular catheterization can also be used to measure pressures throughout the four chambers of the heart and evaluate pressure differences across the major heart valves. In further applications, cardiovascular catheterization can be used to estimate the cardiovascular output, or volume of blood pumped by the heart per minute.

Some medical procedures may require catheterization into the left atrium of the heart. For this purpose, to avoid having to place a catheter in the aorta, access to the left atrium is generally achieved by accessing the right atrium, puncturing the interatrial septum between the left and right atria of the heart, and threading the catheter through the septum and into the left atrium. Cardiovascular puncture must be conducted with extreme precision, as accidental puncturing of surrounding tissue may cause profoundly acute damage to the heart. In addition, cardiovascular puncture may require complicated instruments which are not helpful in guaranteeing the precision of the puncture.

The use of devices available today present many challenges for doctors attempting to puncture the interatrial septum and perform cardiovascular catheterization. Locating the interatrial septum, properly placing the distal end of the puncturing device at the desired location of the septum, safely puncturing the interatrial septum, avoiding accidental punctures, and tracking and maneuvering the catheter post-puncture, are among the many challenges facing those performing cardiovascular catheterization today. Successful outcomes have been achieved by providing balloon-tipped cardiovascular insertion devices as disclosed in US Pub. Nos. US2019/0029722A1, US2019/0029750A1, US2020/0297412A1, and US2021/0100981A1, all to Maini. Additional improvements have resulted from providing steerable cardiovascular insertion devices as disclosed in U.S. Ser. No. 16/897,472 to Maini. And positive results are achieved utilizing a novel handle as disclosed in U.S. Pub. No. 2021/0100981 to Maini. The disclosures of each of these applications and publications are incorporated by reference.

### SUMMARY

Therefore, it is an object of the present disclosure to provide a cardiovascular insertion device to achieve safe, accurate and effective access to tissue, such as various cardiovascular structures, including, for example, cardiac and peripheral vascular structures. The cardiovascular insertion device includes a multichambered device port on its proximal end and a positioning balloon on its distal end with a sheath defining a sheath lumen extending therebetween. Upon achieving vascular or other access to the tissue for treatment, the sheath lumen provides access for medical interventions.

It is an object of the present disclosure to provide a cardiovascular insertion device port comprising a multichambered device port including a plurality of hermetically sealed chambers including: a distal balloon inflation chamber, a medial flush chamber, and a proximal hemostasis valve chamber. The distal balloon inflation chamber includes a balloon port defined by the sheath in fluid communication with a balloon conduit which extends from within the distal balloon chamber and exterior to the multichambered device housing for providing fluid to and from the balloon for inflation and deflation. The medial flush chamber includes a flush port, defined by the multichambered device port main body, in fluid communication with a flush conduit which extends from within the medial flush chamber and exterior to the multichambered device housing for lushing the sheath lumen after or during medical treatment. The proximal valve chamber houses a hemostasis valve in a sealed chamber.

It is a further object of the present disclosure to provide a balloon port lumen for communicating with the hermetically sealed balloon chamber and for inflating the balloon on the distal end of the cardiovascular insertion device. The balloon port lumen extends from the distal balloon chamber along the sheath, to a distal balloon port positioned within the balloon. The balloon lumen extends between sheath layers. The sheath includes an inner layer, a braid layer, and an outer layer. The balloon lumen extends within the braid layer wherein braids wrapped in one direction extend over the balloon lumen and braids wrapped in another direction (thus forming a matrix) extend below the balloon lumen for securing the lumen on the inner layer and preventing shifting of the lumen.

It is a further object of the present disclosure to provide a pull wire port lumen for flexing or bending the distal end of the cardiovascular insertion device for precise positioning. At least one pull wire lumen extends within the braid layer similar to the balloon lumen along the length of the sheath. The pull wire enters the braid layer distal to the balloon lumen and terminates on the distal end of the cardiovascular insertion device at a collar proximal to the balloon lumen distal port. These and other objects are met by the embodiments as shown and described herein.

### BRIEF DESCRIPTION OF THE DRAWINGS

The preferred embodiments described herein and illustrated by the drawings hereinafter be to illustrate and not to limit the invention, where like designations denote like elements.

FIG. 1 is a perspective view of the cardiovascular insertion device having a handle and multichambered device port on its proximal end, a balloon in its distal end, and a sheath extending therebetween;

FIG. 2 is a perspective view of the multichambered device port connected to the sheath;

FIG. 3 is a perspective view of the multichambered device port with the housing removed;

FIG. 4 is a cross sectional view of the multichambered device port taken longitudinally along the axis of the sheath;

FIG. 5 is a partially cut-away view of the multichambered device port wherein the outer housing is partially cut-away to expose the multichambered device port;

FIG. 6 is a cross sectional view of the multichambered device port connected to a device handle, also shown in cross section;

FIG. 7 is a cross sectional view of the device handle and the multichambered device port;

FIG. 8 is a top plan view of the device handle and the multichambered device port with the outer housing top portion removed;

FIG. 9 is a perspective view of the distal and of the cardiovascular insertion device showing the balloon inflated;

FIG. 10 is cross-sectional view of the sheath distal end and the inflated balloon taken longitudinally along the axis of the sheath showing the distal end of the cardiovascular insertion device;

FIG. 11 is cross-sectional view of the cardiovascular insertion device distal end and the inflated balloon taken longitudinally along the axis of the sheath showing the distal end of the cardiovascular insertion device with the outer layer removed;

FIG. 12 is a cross-sectional view of the sheath distal end and the inflated balloon taken radially, perpendicular to the axis of the sheath showing the distal end of the cardiovascular insertion device with the outer layer removed;

FIG. 13 is a cross sectional view taken along 13-13 in FIG. 8 illustrating the collar on the sheath distal end;

FIG. 14 is a cross-sectional view of the sheath taken along 14-14 in FIG. 12 adjacent a proximal portion showing the pull wires entering the sheath layers;

FIG. 15 is a perspective view of the inner layer, braided layer and balloon lumen of the sheath;

FIG. 16 is a perspective view of the inner layer, braided layer and pull wire lumen on the proximal portion of the sheath;

FIG. 17 is a perspective view of the inner layer, balloon lumen, pull wire lumen and braid layer adjacent the proximal end of the sheath; and

FIG. 18 is a schematic, perspective view of the cardiovascular insertion device distal end showing the distal ends of the braid layer, balloon lumen, pull wire lumen and distal end of device with layers shown as transparent for discussion purposes.

### DETAILED DESCRIPTION

The present disclosure will now be described in detail hereinafter by reference to the accompanying drawings. The invention is not intended to be limited to the embodiments described; rather, this detailed description is provided to enable any person skilled in the art to make and practice the invention.

As used herein, the singular forms “a,” “an” and “the” include plural referents unless the context clearly dictates otherwise. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” and “select” or “selectively” mean that the subsequently described event or circumstance may or may not occur, and that the description includes instances where said event or circumstance occurs and instances where it does not. For the purposes of describing and defining the present invention it is noted that the use of relative terms, such as “substantially,” “generally,” “approximately,” and the like, are utilized herein to represent an inherent degree of uncertainty that is attributed to any quantitative comparison, value, measurement, or other representation. These terms are also utilized herein to represent the degree by which a quantitative representation may vary from a stated reference without resulting in a change in the basic function of the subject matter at issue. The term “proximal” as used herein refers to the medical device end closest to the practitioner or use, “distal” refers to the end of the medical device for contacting tissue for medical treatment, “longitudinal” direction refers to the direction between the proximal and distal ends, and the “radial direction” refers to a direction opposite the longitudinal axis, at any angle. As used herein, “fluid” refers to either a liquid or a gas.

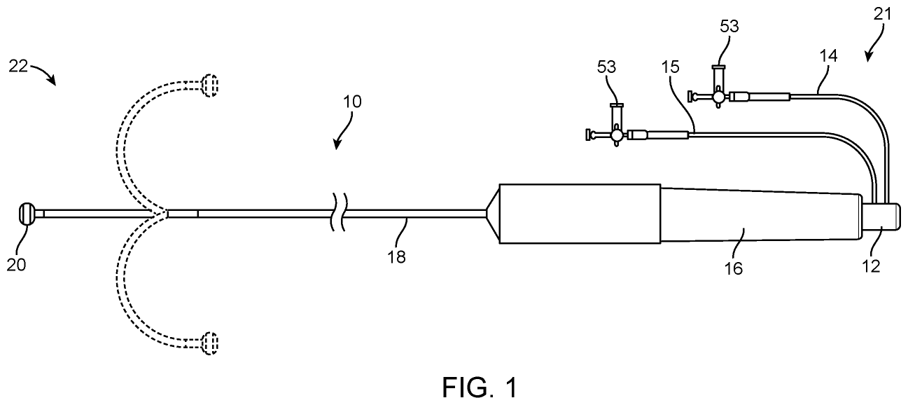

FIG. 1 illustrates the cardiovascular insertion device 10 which includes a proximal end 21 and a distal end 22. Adjacent the proximal end 21 is handle 16 and proximal to the handle 16 is the multichambered device port 12. Extending from the multichambered device port 12 are at least one conduit, or as shown two conduits: balloon conduit 15 and flush conduit 14 which extend radially outwardly from the multichambered device port 12. Sheath 18 extends from the multichambered device port 12, through the handle 16 and to a location adjacent to the cardiovascular insertion device 10 distal end 22 providing the cardiovascular insertion device 10 with a center lumen providing access to anatomical structures for medical interventions. Sheath 18 defines a sheath lumen 19 extending between its proximal and distal ends. Supported on the distal end of the cardiovascular insertion device 10 is the balloon 20.

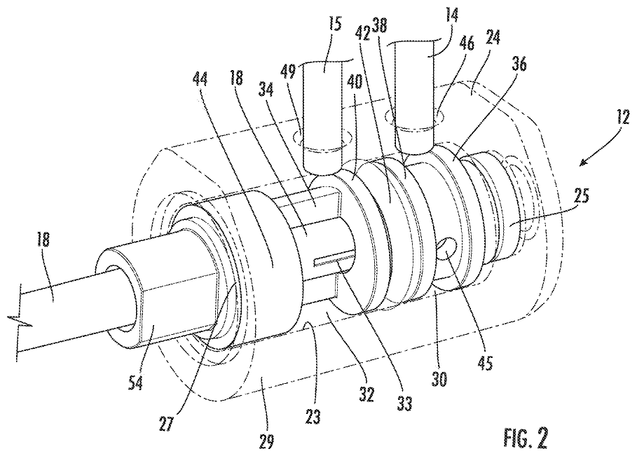

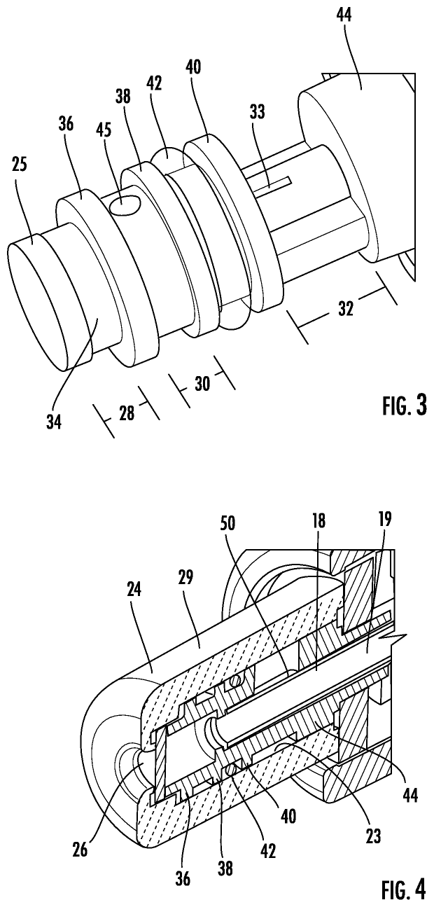

The multichambered device port 12 is shown in perspective view within a multichambered device port housing 24 in FIG. 2 and without the housing 24 in FIG. 3. The housing 24 cooperates with various radially extending surfaces of the multichambered device port 12 to create hermetically sealed chambers within the housing 24. As shown in FIG. 2 and other Figures, the housing has a predetermined thickness defined between the housing outer wall 29 and housing inner wall 23. The housing 12 defines a proximal aperture 26 best shown in FIG. 4. A hemostasis valve 25 is positioned on the proximal end of the multichambered device port 12 and is positioned to obfuscate and seal the housing proximal aperture 26 and to adjust/equalize pressure within the sheath lumen 19. The housing 12 includes a distal aperture 27 configured for receipt of and to seal with the multichambered device port 12 against fluid flow between the inside of the multichambered device port 12 and the outside of the multichambered device port 12.

The multichambered device port is hermetically sealed and includes at least one and, as shown, three hermetically sealed chambers: proximal chamber 28 or valve chamber 28; medial chamber 30 or flush chamber 30; and distal chamber 32 or balloon chamber 32 each of which cooperate with a housing 24. Each of these chambers are hermetically sealed from each other and the outside due to the member of the multichambered device port 12 cooperating with the housing 24 wherein predetermined tolerances and clearances are provided to form hermetic seals. The multichambered device port 12 includes a main body 34 having a central lumen 35 configured for receipt of the sheath 18 and a distal end configured to cooperate with the valve 25. As such, the valve 25 is aligned with the longitudinal axis of the sheath lumen 35 wherein the valve 25 may be pierced by a dilator or the like to access the sheath lumen 35 for medical intervention, such as to catheterized tissue for treatment.

The multichambered device port 12 includes a first flange 36 which extends circumferentially around the device port main body 34. First flange 36, the valve 25 (closing the proximal aperture 26) and proximal end of the housing 24, and the housing inner wall 23 are configured to provide the hermetically sealed proximal chamber 28. Second flange 38, together with first flange 36 and the housing inner wall 23, cooperate to define the medial sealed chamber 30. An O-ring 42, formed by way of example of an elastomeric material, such as silicone, is positioned between the second flange 38 and third flange 40 to ensure a seal between the medial chamber 30 for flushing and the distal chamber 32 for balloon 20 inflation. On its distal side, the third flange 40 cooperates with the housing inner wall 23 and port housing distal member 44 to define the distal chamber 32. The flanges 36, 38, 40 and distal member 44 extend radially outwardly from the main body 34 a distance less than or equal to the diameter of the housing inner wall 23 such they abut against the inner wall 23 of the housing 24 to hermetically seal the chambers 28, 30, 32.

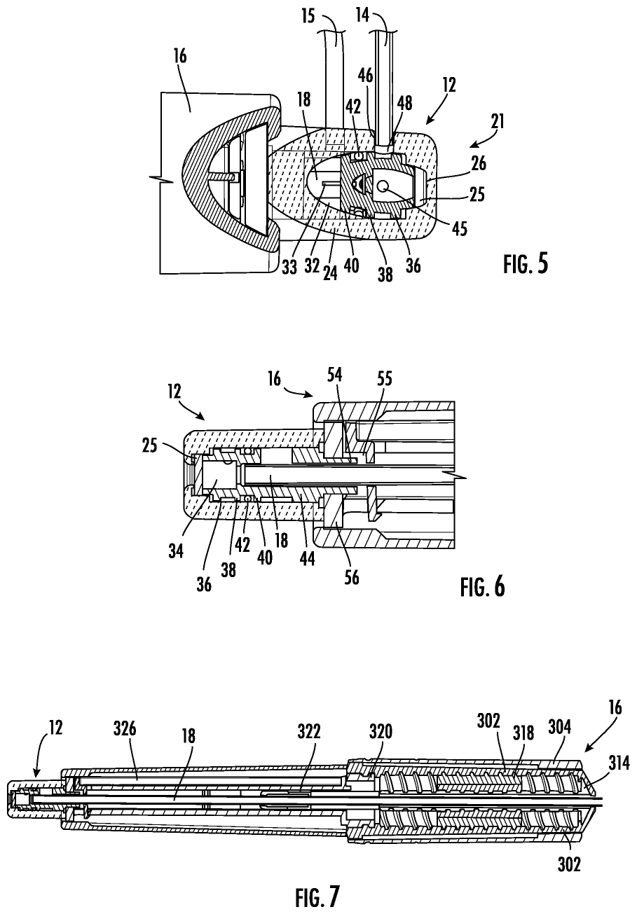

The proximal chamber 28 is hermetically sealed and houses the valve 25 as shown in FIGS. 2, 4, and 5. With reference to FIG. 5, the housing 24 has a proximal end which defines the proximal aperture 26 and which is configured to securely receive the valve 25 within a concave portion. The sealed proximal chamber 28 thus prevents fluid communication with the adjacent medial chamber 30 for flushing the sheath 18. The sealed proximal chamber 28 defines an open interior as shown in FIG. 4 providing access to the sheath 18 proximal end and the sheath lumen 19 such as by a dilator which pierces the valve 25 to access the sheath lumen 19 to achieve vascular access for treatment.

The medial chamber 30 provides a chamber for flushing the sheath lumen 19. As best shown in FIGS. 2-5, within the confines of the medial chamber 30, the device port main body 34 defines at least one flush port 45. According to one aspect, two flush ports 45 are defined by the main body 34 and, as shown, are diametrically opposed on the sides of the main body 34. Of course, any number of ports may be provided. The inner wall 23 of the port housing 24 defines a flush access port 46 extending though the port housing 24. The flush conduit 14 extends within the flush access port 46 to achieve fluid communication with the medial chamber 30 for flushing the medial chamber 30. According to the aspect shown, a sealing membrane 48 is provided to seal the connection of the flush conduit 14 and the housing 24. At least in part because the medial chamber 30 is sealed, the location of the flush port(s) 45 may vary. For example, the flush port 45 may alternatively communicate directly with the flush conduit 14 rendering the hermetic seal of the medial chamber 30 optional.

The distal chamber 32 provides a chamber for providing fluid to the balloon lumen 52 for inflating the balloon 20 and for permitting fluid exit from the sheath lumen 52 such as for deflating the balloon 20. As shown, the sheath 18 extends from the medial chamber 30. The sheath 18 defines a balloon port 50 of the balloon lumen 52 on its exterior end surface for providing fluid communication between the distal chamber 32 and the balloon lumen 52 as described in more detail below. The balloon conduit 15, shown in the form of tubing, extends from the exterior of the port housing 24, through an access port 49 extending through the port housing 24 wall and is in fluid communication, such as having an open end, with the distal chamber 32 which is hermetically sealed. Thus, fluid may enter the distal chamber 32 from the balloon conduit 15 and enter the balloon port 50. It is within the scope of the present disclosure, however, that the balloon port 50 may alternatively communicate directly with the balloon conduit 15 rendering the hermetic seal of the medial chamber 30 optional. Fluid may also exit the sheath lumen 52 through the balloon port 50 and through the balloon conduit 15 such as for deflating the balloon. Any number of balloon ports 50 may be provided. As shown by way of example, the distal chamber 32 comprises one balloon port 50 on the sheath 18. A sealing membrane 48 is optionally provided, as shown, to enhance the integrity of the balloon conduit 15 connection of the housing inner wall 23. The flush conduit 14 and the balloon conduit 15 include respective valves 53 for controlling fluid flow of, in both directions, configured to cooperate with a fluid source for, and for providing access to the conduits 14, 15.

According to one aspect, the multichambered device port 12 is connected at its distal end to a handle assembly 16. The sheath 18 also extends through, such as completely through or at least halfway through, the length of the handle assembly 16. The handle assembly 16 shown herein may be used with the disclosure of U.S. Pub. No. 2021/0100981 to Maini, the disclosure of which is hereby incorporated by reference. This is an exemplary handle assembly which optionally may be used with the multichambered device port 12 and sheath 18 according to some embodiments. It is to be appreciated, however, that use of other handle assemblies, or no handle assembly, is within the scope of the present disclosure. For the sake of discussion, this exemplary handle assembly 16 is shown and described.

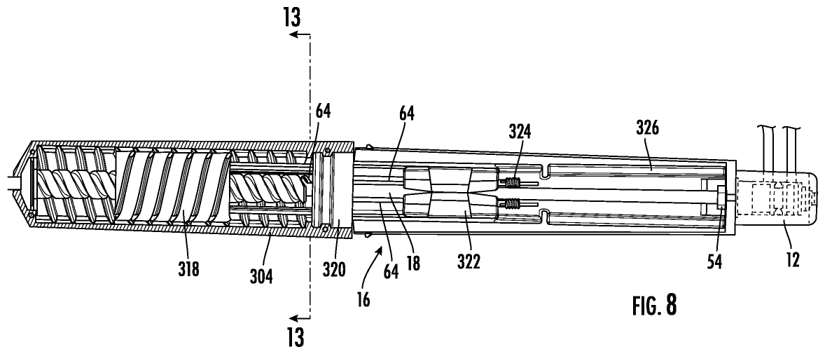

With reference to FIGS. 7 and 8 wherein FIG. 7 is a cross-section of the handle 16 and FIG. 8 is a top perspective with outer layers removed, shown are two twister thread half shells 302. Also shown is grip overmold 304. As shown, handle 16 also includes twister bolt inner 314, a nut inner 316, a nut outer 318, a handle inner 320, a coupler 322, a wire clamp assembly 324, and a handle inner cover 326. Nut inner 316 and nut outer 318 are each connected to one of two pull wires 64 that extend through the handle 16 and attached to different opposite sides of distal end of the sheath 18 as shown in FIGS. 7-8 and may be oriented about the circumference of the distal end of the sheath at locations 180 degrees apart. As shown in FIG. 8, the pull wires 64 enter the sheath 18 layers as described in more detail below. Nut inner 316 and nut outer 318 are rotated in different, opposite directions by turning of the twister thread half shells 302. Twister thread half shells 302 have internal threads that engage with threads of nut outer 318. Nut outer 318 has an alignment key that engages the nut inner 316 and allows nut inner 316 to move linearly, sliding against the nut outer 318. The internal thread of the nut inner 316 engages with the twister bolt inner 314. The twister bolt inner 314 is locked in engagement with the twister half shell 302. Both twister half shells 302 and the twister bolt inner 314 turn together, simultaneously engaging the outer nut 318 and inner nut 316, which move in opposite directions when the group 304 and/or the half shells and/or proximal end of the handle are rotated with respect to the distal end of the handle. When twister thread half shells 302 are rotated in a first direction, nut inner 316 rotates and pulls the pull wire, to which the nut inner 316 is coupled by wire clamp assembly 324, towards the proximal end of handle 16. This movement causes the side of the distal end of sheath 18, to which nut inner 316 pull wire is attached, to bend or flex in a first direction, such as in a direction towards the pull wire towards proximal end of the cardiovascular insertion device 10. Rotating twister thread half shells 302 in a second direction causes the nut outer 318 to rotate and pull the pull wire, to which the nut outer 318 is coupled by wire clamp assembly 324, towards the proximal end of handle 16. This movement causes the side of the distal end of sheath 18, to which nut outer 318 pull wire is attached, to bend or flex towards in a second direction, such as in a direction towards the pull wire proximal end. In some embodiments, the distal end of the sheath 18, may rotate with up to or at least 180 degrees of flex or turn, see for example FIG. 1. In some embodiments, the distal end of the sheath 18, may rotate up to or at least 270 degrees of flex or turn. When nut inner 316 and nut outer 318 so rotate, the nut inner 316 and the nut outer 318 each slides in the opposite direct along their smooth adjoining surfaces.

It is to be appreciated, however, that the handle 16 shown and described herein is exemplary as the multichambered device port 12 and the sheath 18 are beneficially compatible with other handles. As shown, the multichambered device port 12 cooperates with the handle 16 and is connected to the handle 16 proximal end. As shown in FIGS. 4 and 6-8, the sheath 16 extends though the handle 16 and into the multichambered device port 12. In some embodiments the multichambered device port 12 and the handle 16 may connect mechanically with corresponding interlocking members and/or with the benefit of adhesives on mating surfaces. As shown, the distal most portion of the multichambered device port 12 defines locking member 54 which is configured to be received by receiving member 55 defined by the handle 16. A ring 55 on the proximal end of the handle 16 provides a surface by which adhesive may optionally be applied to further secure the interlocking connection between the multichambered device port 12 and the handle 16.

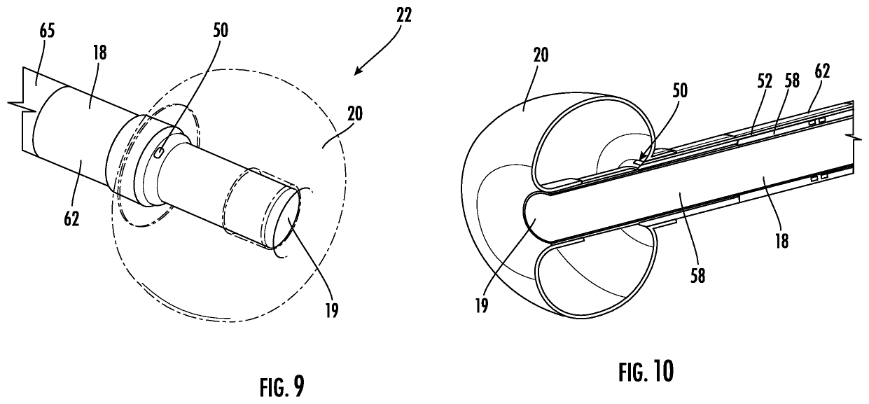

FIGS. 9-12 illustrate the distal end 22 of the cardiovascular insertion device 10. As shown, the cardiovascular insertion device 10 distal end 22 includes a positioning balloon 18 which extends distally from the cardiovascular insertion device 10 and overhangs the cardiovascular insertion device 10 for positioning the cardiovascular insertion device 10 for implantation, penetration, and/or treatment. Exemplary balloons are as set forth in US Pub. Nos. US2019/0029722A1, US2019/0029750A1, US2020/0297412A1, and US2021/0100981A1, all to Maini. These are incorporated herein by reference.

In some embodiments, a balloon lumen 52 may extends longitudinally along the length of the cardiovascular insertion device 10, between at least two layers of the sheath 18. The sheath 18 includes at least two, or as shown, three layers: inner layer 58; intermediate or braid layer 68, and outer layer 62. The balloon lumen 52 extends from distal chamber 32 of the multichambered device port 12 and extends along the length of the sheath 18, between the inner layer 58 and braid layer 68. The balloon lumen 52 extends into the balloon 20 with a distal balloon port 50 on its distal end. As shown in FIGS. 9-12, the distal balloon port 50 is positioned within the balloon 20 which, as shown, extends around a distal end of the cardiovascular insertion device 10. To inflate the balloon 20, the balloon conduit 15 introduces fluid into the distal chamber 32, the fluid enters the proximal balloon port 33, the fluid advances along the length of the balloon lumen 52 within layers of the sheath 18 and the distal end of the cardiovascular insertion device 10 and exits the distal balloon port 50 positioned inside the balloon 20 to inflate the balloon 20. Deflation of the balloon 20 is achieved in the reverse process: though the distal balloon port 50, along the balloon lumen 52; into the distal chamber 32 via the proximal balloon port 33 and exits through the balloon conduit 15.

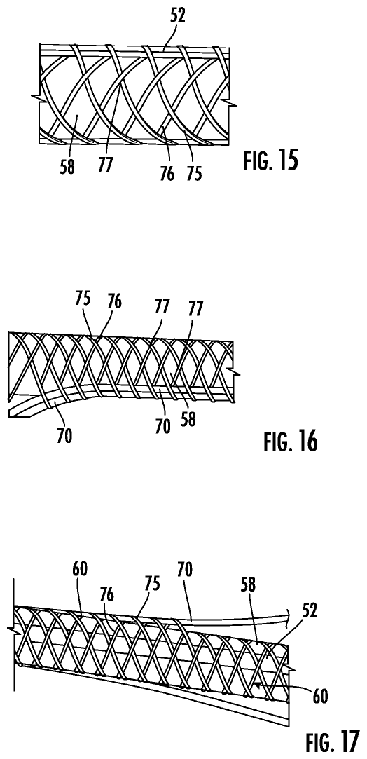

The balloon lumen or lumens 52 extend between adjacent layers of the sheath 18. As previously described, the balloon lumen 52 proximal end is within the distal chamber 32. The balloon lumen 52 extends along the inner layer 58 of the sheath as shown in FIG. 15. The balloon lumen 52 may be intertwined within the braid layer 60. The braid layer 60, according to some embodiments, may include a first braid 75 wrapped around the inner layer 58 in a first direction which is at an angle relative to the longitudinal axis, such as between 30° to 90° to the longitudinal axis of the sheath 18 and, more specifically, between 40° and 50°, such as 45°. The braid layer 60 may also include a second braid 76 wrapped around the inner layer 58 in a second direction which is at an angle relative to the longitudinal axis, such as between 30° to 90° to the longitudinal axis of the sheath 18 and, more specifically, between 40° and 50°, such as 45°. The first 75 and second 76 braids intersect at matrix intersections 77. As shown in FIG. 15, the balloon lumen 52 extends along the braid layer 60 between the first 75 and second braid 76 at points of intersection 77. Thus, one of the braids, e.g., first braid 76, extends beneath the balloon lumen 52 and other braid, e.g., second braid 76, extends over the balloon lumen 52. Of course, these may be reversed.

While not intending to be bound by any particular theory, it is believed that the braid matrix formed of the first 75 and second braids 76 secures the balloon lumen 52 due, at least in part, to frictional forces resulting from the braid matrix and the materials selected. Shifting of the balloon lumen 52 in the radial direction (that is, “around” the inner layer 58) is precluded by this novel matrix configuration. The inner layer is formed of a formable or extruded material, such as stainless steel, and the braid layer 60 is formed of a formable or extruded material, such as stainless steel. The outer layer 62, formed of an elastomeric material such as an elastomer sold under the brand Pebax®, surrounds the braid layer 60 and balloon lumen 52. The first braid 75 and second braid 76 are shown with the same width and thickness but in some embodiments one or both may be non-uniform.

In some embodiments, pull wires 64 are provided to flex or bend the sheath 18 distal end as described above. Similar to the balloon lumen 52, pull wire lumens 70 configured for receipt of the pull wires 64, are provided within the braid layer 60. As shown in FIG. 8, the pull wires 64 originate proximally in the handle 16 and enter the sheath distal to the handle inner 320. FIG. 14 illustrates the pull wire 64 entering the sheath 18 through an aperture therein. The pull wire 64 enters the pull wire lumen 70 within the braid layer 60 as shown in FIG. 16. FIG. 16 shows the pull wire lumen 70 entering the braid layer 60 and being positioned between the first 75 and second 76 braids similar to the balloon lumen 52. FIG. 16 illustrates the entry of the pull wire lumen 52 into the braid layer 60.

FIG. 17 illustrates the sheath 18 with the inner layer 58 and braid layer 60 and including both the pull wire lumens 70 and balloon lumen 52 at the proximal location wherein the pull wire lumen 70 enters the braid layer 60. As shown, the balloon lumen 52 is positioned within the braid layer 60 at a more proximal location than the pull wire lumen 70 (wherein it originates proximally in the distal chamber 32).

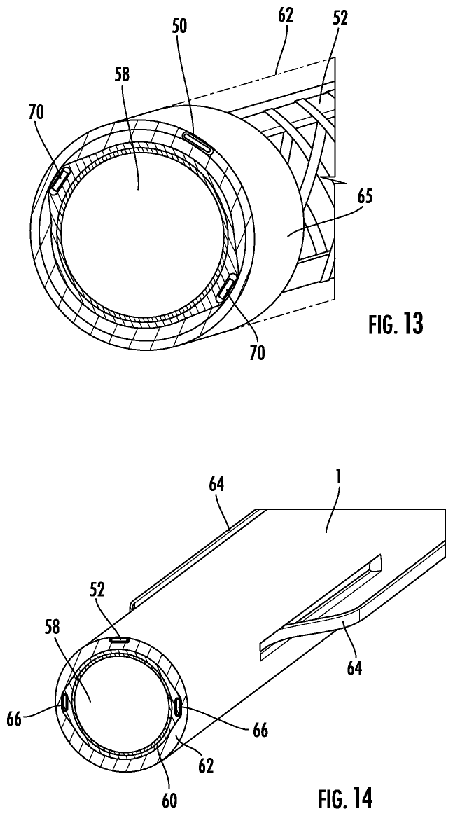

On the distal end of the sheath 18, the pull wire lumen 70 terminates distally at a collar 65. FIG. 13 is a cross sectional view taken along 14-14 in FIG. 12 at the distal end of the collar 65. Shown in FIG. 13 are the distal ends of the pull wire lumens 52 and an interim portion of the balloon lumen 50 (which continues distally along the device 10). At least one pull wire 64 is provided and, by way of example, two pull wires 64 are shown in the Figures. The pull wires 64 extend from the handles 16 as previously described, enter the sheath 18 as shown in FIGS. 8 and 13-14, and extend along the length of the sheath 18 between the inner layer 58 and the braid layer 60 and terminating at the collar 65. The collar 65 is configured to provide structural integrity to the portion of the sheath 18, adjacent its distal end but proximal to the balloon 20. The collar 65 is formed of a formable or extrudable material, such as stainless steel.

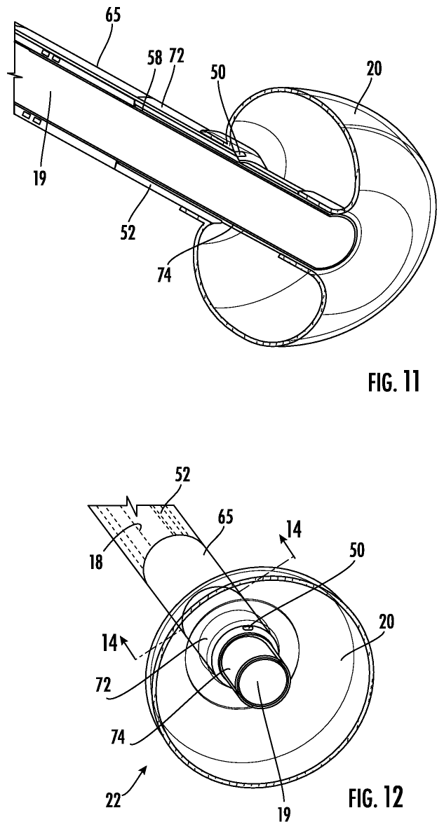

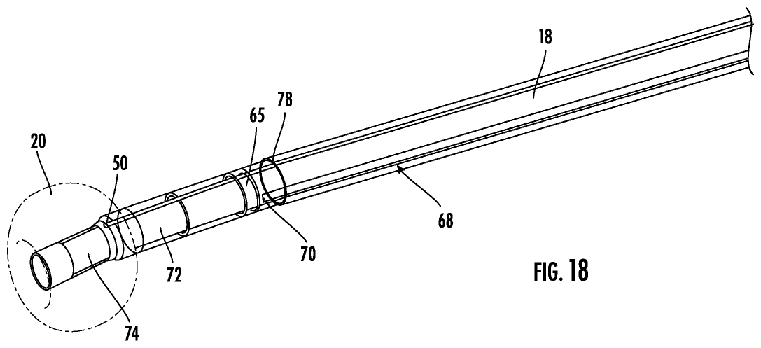

In some embodiments, for example, as shown in FIGS. 9-12 and 18, the sheath 18 extends distally to a collar 65 which is placed around the sheath 18 and the pull wire lumens 70 which terminate in the collar 65. FIG. 18 illustrates the distal ends of the various members and the members of the cardiovascular insertion device 10 some of which are shown with transparency for discussion purposes only wherein the members are not transparent in actuality. The braid layer 60, according to an aspect shown in FIG. 18, terminates proximal to the collar 65 at location labelled 78. A first distal shaft 72 connects to the sheath 18 adjacent the collar 65 and a second distal shaft 74 extends distally from first distal shaft 72 and into the balloon 20. As shown in FIG. 18, the balloon lumen 52 extends along the first distal shaft 72 and the balloon port 50 is within the balloon 20 adjacent the second distal shaft 74. This is also shown in FIG. 11. This is beneficial, at least, to facilitate flexing of the cardiovascular insertion device 10. The pull wire lumens 70 via pull wires 64 exert forces (explained above) on the collar 65 as the handle 16 is manipulated. Flexing or bending of the cardiovascular insertion device 10 occurs between the collar 65 and the distal end of the braid layer 68 which ends distally at its distal end 78 shown in FIG. 18. The lack of the braid layer 68 immediately proximal to the collar 65 facilitates flexing in that region.

The cardiovascular insertion device 10 thus provides vascular access to tissue, such as cardia chambers and other vascular beds or structures including arterial, venous, or other tissue. Upon proper positioning of the cardiovascular insertion device 10 (utilizing the pull wires 64 where necessary) and the balloon 20 is inflated (utilizing the multichambered device port), the sheath lumen 19 is positioned and available for treatment. Once access is achieved, sheath lumen 19 provides vascular access to other devices as necessary for treatment. Upon completion of positioning, the balloon 20 is deflated (by way of the balloon lumen 52 and the distal chamber 32). Upon completion of treatment, the medial chamber 30 and flush conduit 14 permit flushing of the lumen 18 of the cardiovascular insertion device 10. The method of gaining vascular and/or other arterial and venous access elsewhere in the body includes the step of inserting the cardiovascular insertion device 10 into the vasculature; selectively utilizing the handle 16 (according to some embodiments) and pull wire 64 to position the distal end 22 of the cardiovascular insertion device 10 against the surface to be treated (for example, penetrated); inflating the balloon 20 cardiovascular insertion device 10 utilizing the balloon conduit 15; achieving access through the sheath lumen 19 of a medical device for treatment; deflating the balloon 20 utilizing the balloon lumen 52, distal chamber 32, and balloon conduit 15; and treating targeted tissue.

While exemplary embodiments have been shown and described above for the purpose of disclosure, modifications to the disclosed embodiments may occur to those skilled in the art. The disclosure, therefore, is not limited to the above precise embodiments and that changes may be made without departing from its spirit and scope.

1. A cardiovascular insertion device, the device comprising:
a body having a proximal end and a distal end;
a multichambered device port at the proximal;
a positioning balloon at the distal end; and
a sheath extending between said proximal and distal ends and defining a sheath lumen;
wherein the multichambered device port includes:
a first chamber in fluid communication with the positioning balloon, and
a second chamber in fluid communication with the sheath lumen.

2. The cardiovascular insertion device of claim 1, wherein:
the first chamber and the second chamber are hermetically sealed from each other.

3. The cardiovascular insertion device of claim 1, further comprising:
a balloon lumen extending within a sidewall of the sheath from the first chamber to the balloon.

4. The cardiovascular insertion device of claim 3, wherein:
a proximal end of the balloon lumen includes an opening to the first chamber.

5. The cardiovascular insertion device of claim 3, wherein:
a distal end of the balloon lumen includes an opening to the balloon.

6. The cardiovascular insertion device of claim 1, further comprising:
a housing of the multichambered device port surrounding the first and second chambers;
a first access port through the housing in selectable fluid communication with the first chamber and configured to provide fluid to the balloon.

7. The cardiovascular insertion device of claim 6, further comprising:
a second access port through the housing in selectable fluid communication with the second chamber and configured to provide fluid to the sheath lumen.

8. The cardiovascular insertion device of claim 6, further comprising:
a main body of the multichambered device port within a central cavity of the housing, wherein the main body and the housing define the first and second chambers.

9. The cardiovascular insertion device of claim 8, further comprising:
a port within the main body that couples the second chamber in fluid communication with the sheath lumen.

10. The cardiovascular insertion device of claim 6, further comprising;
an aperture in a proximal end of the housing; and
a hemostatic valve configured to provide access to the sheath lumen via the aperture.

11. The cardiovascular insertion device of claim 1, further comprising;
a handle between the multichambered device port and the distal end,
wherein the sheath extends though the handle to the multichambered device port.

12. The cardiovascular insertion device of claim 11, wherein:
the handle includes a proximal end and a distal end, the proximal end rotatable with respect to the distal end.

13. The cardiovascular insertion device of claim 11, wherein:
the handle includes:
a shell having threads that engage with outer threads of a nut; and
an inner bolt having threads that engage with inner threads of the nut.

14. The cardiovascular insertion device of claim 13, wherein:
the nut includes an outer nut having the outer threads of the nut and an inner nut having the inner threads of the nut.

15. The cardiovascular insertion device of claim 14, wherein:
the inner nut and outer nut are slidingly engaged with each other.

16. The cardiovascular insertion device of claim 15, wherein:
the inner nut is coupled to a proximal end of a first wire, the distal end of the first wire being coupled to the distal end of the sheath at a first location, and
the outer nut is coupled to a proximal end of a second wire, the distal end of the first wire being coupled to the distal end of the sheath at a second location.

17. The cardiovascular insertion device of claim 16, wherein:
rotation of the proximal end of the handle in a first direction with respect to the distal end of the handle causes the inner nut and outer nut to move along a longitudinal axis of the handle relative to each other to pull on a first one of the first wire and the second wire to cause the distal end of the sheath to bend in a first direction.

18. The cardiovascular insertion device of claim 17, wherein:
rotation of the proximal end of the handle in a second direction, opposite the first direction, with respect to the distal end of the handle causes the inner nut and outer nut to move along the longitudinal axis of the handle relative to each other to pull on a second one of the first wire and the second wire to cause the distal end of the sheath to bend in a second direction, opposite the first direction.

19. The cardiovascular insertion device of claim 17, wherein:
the distal end of the sheath bends at least 180 degrees.

20. The cardiovascular insertion device of claim 17, wherein:
the distal end of the sheath bends at least 270 degrees.
